(19) United States
(12) Patent Application Publication
(10) Pub. No.: US 2022/0008787 A1
(43) Pub. Date: Jan. 13, 2022

(54) EVALUATION DEVICE, EVALUATION SYSTEM, AND STORING MEDIUM

(71) Applicant: Toyota Jidosha Kabushiki Kaisha, Toyota-shi Aichi-ken (JP)

(72) Inventors: Yurika Tanaka, Yokosuka-shi (JP); Shuichi Sawada, Nagoya-shi (JP); Shin Sakurada, Toyota-shi (JP); Yasuhiro Baba, Kawabe-chou (JP); Shintaro Matsutani, Kariya-shi (JP); Tomoya Makino, Kariya-City (JP)

(21) Appl. No.: 17/366,617

(22) Filed: Jul. 2, 2021

(30) Foreign Application Priority Data
Jul. 7, 2020 (JP) 2020-116832

(51) Int. Cl. A63B 24/00; G16H 20/30; G06Q 30/02; G16H 10/60; G16H 50/70; G16H 40/67

(52) U.S. Cl. CPC A63B 24/0062; A63B 24/0059; G16H 20/30; G06Q 30/0215; A63B 2230/062; G16H 50/70; G16H 40/67; A63B 2024/0068; A63B 2220/836; G16H 10/60

(57) ABSTRACT

An evaluation device, comprises a controller configured to: obtain exercise data, the exercise data being data related to exercise performed by a user; generate incentive data based on the exercise data, the incentive data indicating incentives to be provided to the user; determine whether an exercise appropriate for the user is being performed, the determining being performed based on the exercise data; and when determining that an exercise appropriate for the user is not performed, adjust an amount of the incentives to be provided to the user.

### CROSS REFERENCE TO THE RELATED APPLICATION

This application claims the benefit of Japanese Patent Application No. 2020-116832, filed on Jul. 7, 2020, which is hereby incorporated by reference herein in its entirety.

### BACKGROUND

### Technical Field

The present disclosure relates to a technique for supporting health promotion for a user.

### Description of the Related Art

There has been a system in which incentives such as points are provided according to exercise amount in order to encourage health promotion. For example, Japanese Patent Laid-Open No. 2019-145140 discloses a system in which an activity performed by a user is sensed and virtual currencies which can be used for purchasing products and services, discounting, and the like are provided to the user based on the amount of the activity.

### SUMMARY

In a situation where incentives are provided according to the amount of activity, user's purpose may change from maintaining health to receiving rewards. In such a situation, adverse effects may occur such as repeating only the exercises through which rewards can be obtained more efficiently and exercising too much for rewards, resulting in damage to health.

It is an object of the present disclosure to provide a technique for supporting health promotion for a user.

The present disclosure in its one aspect provides an evaluation device, comprising: a controller configured to: obtain exercise data, the exercise data being data related to exercise performed by a user; generate incentive data based on the exercise data, the incentive data indicating incentives to be provided to the user; determine whether an exercise appropriate for the user is being performed, the determining being performed based on the exercise data; and when determining that an exercise appropriate for the user is not performed, adjust an amount of the incentives to be provided to the user.

The present disclosure in its another aspect provides an evaluation system, comprising: one or more sensors configured to obtain exercise data, the exercise data being data related to an exercise performed by a user; and a controller configured to: generate incentive data based on the exercise data, the incentive data indicating incentives to be provided to the user; determine, based on the exercise data, whether an exercise appropriate for the user is being performed; and when determining that an exercise appropriate for the user is not performed, adjust an amount of the incentives to be provided to the user.

The present disclosure in its another aspect provides a non-transitory computer readable storing medium recording a computer program for causing a computer to perform an information processing method comprising: a step of obtaining exercise data, the exercise data being data related to an exercise performed by a user; a step of generating incentive data based on the exercise data, the incentive data indicating incentives to be provided to the user; a step of determining, based on the exercise data, whether an exercise appropriate for the user is being performed; and a step of, when determining an exercise appropriate for the user is not being performed, adjusting an amount of the incentives to be provided to the user.

In addition, another aspect is an information processing method executed by the above evaluation device or computer program stored in the above non-transitory computer readable storage medium.

According to the present disclosure, a technique for supporting health promotion for a user can be provided.

### BRIEF DESCRIPTION OF THE DRAWINGS

FIG. 1 is a diagram for describing an overview of an exercise evaluation system;

FIG. 2 is a diagram illustrating in details components of an exercise evaluation system according to a first embodiment;

FIG. 3 is a diagram for describing data that is transmitted and received between devices;

FIG. 4A is an example of personal data that is stored in a storage unit;

FIG. 4B is an example of evaluation data that is stored in the storage unit;

FIG. 4C is an example of provision reference data that is stored in the storage unit;

FIG. 5 is a diagram for describing processing that is executed by a control unit;

FIG. 6 is a flowchart of processing that is executed by the control unit in the first embodiment;

FIG. 7A is an example of log data that is generated by the control unit;

FIG. 7B is an example of log data that is generated by the control unit; and

FIG. 8 is a flowchart of processing that is executed by the control unit in the first embodiment.

### DESCRIPTION OF THE EMBODIMENTS

There is known a technique for providing incentives such as points or virtual currencies to a user based on the amount of exercise performed by the user. For example, there can be a service in which calories burned are calculated based on data obtained from a wearable computer and points according to the calories burned are provided.

However, such a service may change user's purpose from maintaining health to obtaining incentives. For example, such cases may occur as repeating only an easy exercise menu in order to efficiently obtain points and selecting an exercise menu which is not appropriate for himself/herself in order to obtain more points. In this case, not only merits on health for the user may be lost, but also their health may be damaged due to overworking, for example.

An evaluation device according to an embodiment is a device that provides incentives to a user based on an exercise performed by the user, which adjusts the incentives when an exercise inappropriate for the user is performed.

More specifically, it includes a control unit that performs: obtaining exercise data that is data related to an exercise performed by a user; generating incentive data that indicates incentives to be provided to the user based on the exercise data; determining whether an exercise appropriate for the user is being performed, based on the exercise data; and when determining that exercise appropriate for the user is not being performed, adjusting the amount of incentives to be provided to the user.

The exercise data can be obtained, for example, based on a result of sensing the user performing exercise. The exercise data may be generated based on data which is obtained from a training machine or fitness machine or may be generated based on data which is obtained from a wearable device (smart watch or the like) worn by the user.

The control unit calculates incentives to be provided to the user, based on the exercise data. The incentives may be, for example, points, virtual currencies, discount coupons, or the like that can be used in online services as long as they are beneficial to the user.

In addition, the control unit determines whether an exercise appropriate for the user is being performed, based on the exercise data. An appropriate exercise for each user can be determined based on, for example, a user profile and the like. In addition, when it is determined that the exercise amount or the like deviates from a proper range, adjustment processing such as reducing the amount of incentives so as to be less than its standard or stopping provision, for example, is performed.

This configuration allows a user to be encouraged to adopt an exercise menu tailored to each individual.

In addition, the evaluation device may further include a storage unit that stores evaluation data for evaluating an exercise performed by the user and the control unit may determine whether at least any one of an exercise intensity, an exercise amount, and an exercise type is appropriate for the user, based on the evaluation data.

Furthermore, the storage unit may further store user information that is information on the user and the control unit may determine an exercise intensity, an exercise amount, or an exercise type that is appropriate for the user, further based on the user information.

The exercise intensity and the exercise amount can be represented by, for example, indices such as metabolic equivalents (METs) and calories burned, respectively. Such indices have different proper ranges according to the age, gender, weight, and the like of a user. Therefore, by using evaluation data associated with a user, a determination suitable for each individual can be made.

In addition, when determining that the exercise intensity or the exercise amount deviates from a proper range for the user, the control unit may reduce a provision rate of incentives to be provided to the user.

If the exercise intensity or the exercise amount deviates from a proper range, effects from the exercise may not be obtained or health may be damaged. In such a case, by reducing the provision rate of incentives (for example, the amount of points to be provided for a predetermined exercise amount), a motivation to perform an appropriate exercise can be given to the user.

In addition, when determining that the exercise type is not appropriate for the user, the control unit may reduce the provision rate of incentives to be provided to the user.

Furthermore, when determining that the type of exercise performed for a past predetermined period is not appropriate for the user, the control unit may reduce the provision rate of incentives to be provided to the user.

For example, when a user is performing an exercise of a type that is not beneficial to the user, repeating only an identical exercise menu, or doing too many exercise menus for an elapsed time, the provision rate of incentives may be reduced because the exercise type is assumed to be inappropriate. By using an exercise type as an evaluation reference, it becomes possible to make the user perform appropriate and various exercise menus.

In addition, when determining that exercise appropriate for the user is not being performed, the control unit may notify the user that the amount of incentives is to be adjusted.

For example, it may notify the user that the provision rate of incentives is recovered by improving the exercise contents. This configuration allows the user to be given a motivation to do an appropriate exercise.

In addition, when determining that an exercise appropriate for the user is not being performed, the control unit may suggest, to the user, an exercise menu that allows the adjustment to be avoided.

For example, causes of performed incentive adjustments, such as too few (or too many) types of exercises or too intense (or too weak) exercise, and its solution may be provided to the user. For example, provision of an appropriate exercise intensity, exercise amount, and exercise type, and the like makes it possible for the user to do an appropriate exercise.

The evaluation system according to another embodiment may further include one or more sensors configured to obtain exercise data, the exercise data being data related to an exercise performed by a user.

In addition, the one or more sensors may include a sensor built in a wearable computer associated with the user.

Furthermore, the one or more sensors may include a heart rate sensor; and the exercise data may include an exercise intensity that is estimated based on a heart rate obtained by the heart rate sensor.

By obtaining data from the wearable computer, more accurate data related to an exercise performed by the user can be obtained.

Hereinafter, embodiments of the present disclosure will be described with reference to drawings. Configurations of the embodiments below are illustrative and the present disclosure is not limited to the configurations of the embodiments.

### First Embodiment

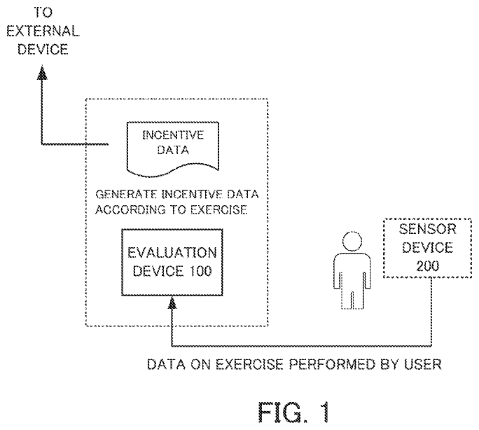

An overview of an exercise evaluation system according to a first embodiment will be described with reference to FIG. 1. The exercise evaluation system according to this embodiment includes: an evaluation device 100 that evaluates an exercise performed by a user; and a sensor device 200 that senses the exercise performed by the user.

The evaluation device 100 is a device that evaluates an exercise performed by the user and provides incentives according to the exercise to the user. In this embodiment, the evaluation device 100 obtains data related to the exercise performed by the user, from the sensor device 200. In addition, it generates incentive data according to the exercise based on the obtained data. The incentive data is data indicating the amount of incentives provided to the user and is transmitted to an external device that manages incentives.

In this embodiment, points that can be used in online services are provided as the incentives to the user.

Note that although the evaluation device 100 corresponds to the user one-to-one in an example of FIG. 1, one evaluation device 100 may manage a plurality of users. In this case, the evaluation device 100 may be configured to be able to communicate with a plurality of the sensor devices 200.

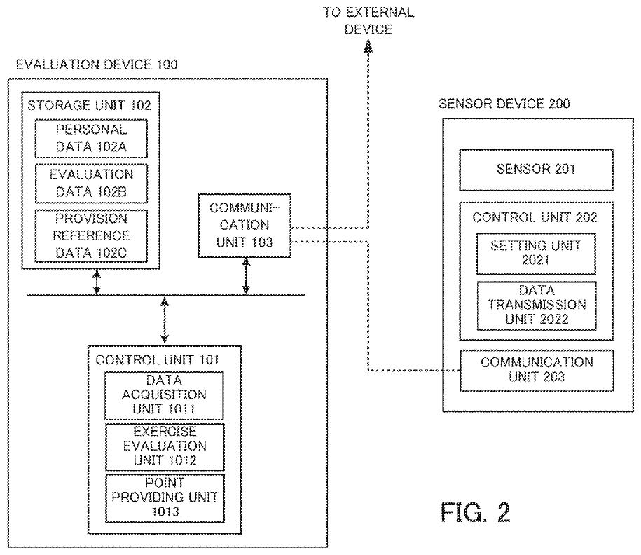

FIG. 2 is a diagram illustrating in more detail components of the exercise evaluation system according to this embodiment. Here, the sensor device 200 will be described first.

The sensor device 200 is a device that senses an exercise performed by a user and outputs information related to the exercise. In this embodiment, the sensor device 200 is exemplified in a mode of being built in a training machine and in a mode of using a wearable computer.

The sensor device 200 is configured with a sensor 201, a control unit 202, and a communication unit 203.

The sensor 201 is a sensor that senses the exercise performed by the user. For example, when the exercise performed by the user is running or cycling, the sensor 201 senses a travel distance thereof. For example, when the sensor device 200 is the one that is built in a training machine or fitness machine (running machine or fitness bike), the sensor 201 obtains the travel distance based on a rotation speed, a load, and the like. In addition, when the sensor device 200 is a wearable computer, the travel distance and an exercise intensity is sensed by a pedometer, a GPS, an acceleration sensor, a heart rate sensor, or the like. Furthermore, when the exercise performed by the user is a muscle training or the like, the sensor 201 may sense the number of movements (for example, the number of abdominal exercises, the number of weight liftings, and the like).

The control unit 202 is a microcomputer that has a function of transmitting a sensing result to the evaluation device 100. The control unit 202 can be configured as, for example, a one-chip microcomputer into which an arithmetic unit, a main memory, and an auxiliary memory are packaged. Part or all of functions of the control unit 202 may be implemented by a hardware circuit such as an ASIC or FPGA.

The control unit 202 is configured with two function modules of a setting unit 2021 and a data transmission unit 2022. Each of the function modules may be implemented by executing a stored program by a CPU.

The setting unit 2021 sets information related to a user who performs an exercise (hereinafter, user information) and information related to the exercise (hereinafter, exercise information).

The user information includes, for example, an identifier of the user (user ID), and the age, gender, and weight of the user. The user information may be set in advance by the user who uses the system. In addition, when the sensor device 200 is the one built in a training machine or fitness machine, the setting unit 2021 may obtain the user information via an external interface (for example, RF tag reader). For example, it is acceptable that a user touches a wrist band having a built-in RF tag, on a reader before starting an exercise and the setting unit 2021 obtains user information based on the tag.

The exercise information includes, for example, the category of exercise (hereinafter, exercise category). Note that when one sensor device 200 targets one type of exercise category, the exercise category may be fixed. In addition, when the sensor device 200 is a wearable computer, an exercise category may be set by the user at the time of starting exercise.

Note that although the term exercise category is used in this embodiment, the exercise category is not limited to running, swimming, weight lifting, and such kinds. For example, categorization according to loads such as a running speed, a swimming speed, and the heaviness of a weight may be performed.

The data transmission unit 2022 generates data that is to be transmitted to the evaluation device 100, based on the set user information and exercise information and sensor data which is obtained by the sensor 201.

The data transmission unit 2022 calculates an exercise intensity (for example, how many times more calories than those in rest time is burned), an exercise amount (for example, the accumulated value of calories burned), the duration of exercise, and the like, based on the user information, exercise information, and sensor data; and transmits them to the evaluation device 100 in association with the user ID. The exercise intensity and calories burned can be calculated based on preliminarily stored data and the set user information.

Note that when the sensor data includes a heart rate, the data transmission unit 2022 may estimate the exercise intensity based on the heart rate. By sensing user's vital information such as a heart rate, more accurate data can be obtained than by using other sensor data (for example a move distance obtained by the GPS).

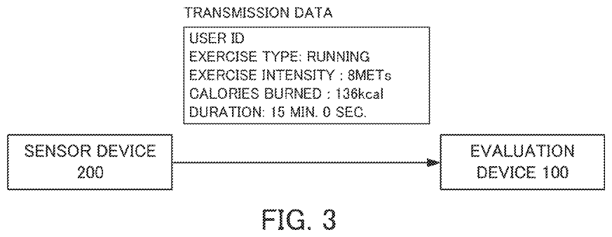

FIG. 3 is an example of data that is to be transmitted from the sensor device 200 to the evaluation device 100.

The communication unit 203 is a communication interface that connects the evaluation device 100 and the sensor device 200. The communication unit 203 includes a wireless communication module, for example.

Next, the evaluation device 100 will be described.

The evaluation device 100 evaluates an exercise being performed by the user, based on the data obtained from the sensor device 200; and generates incentive data indicating incentives (points) that are to be provided to the user, based on a result of the evaluation. The incentive data is transmitted to an external device that manages points. Thus, the user can obtain points according to an exercise result.

The evaluation device 100 can be configured by a general-purpose computer. More specifically, the evaluation device 100 can be configured as a computer that includes a processor such as a CPU, GPU, or the like, a main memory such as a RAM, ROM, or the like, and an auxiliary memory such as an EPROM, hard disk drive, removable media, or the like. Note that examples of removable media may include USB memories and disk recording media such as CD and DVDs. In the auxiliary memory, an operating system (OS), various programs, various tables, and the like are stored, and programs stored therein are loaded into a work area of the main memory and are executed. Through the execution of the programs, each component and the like are controlled and thereby, each function corresponding to a predetermined purpose as described later can be implemented. However, part or all of the functions may be implemented by a hardware circuit such as an ASIC or FPGA.

The control unit 101 is an arithmetic unit that performs control in the evaluation device 100. The control unit 101 can be implemented by an arithmetic processing unit such as a CPU.

The control unit 101 is configured with three function modules of a data acquisition unit 1011, an exercise evaluation unit 1012, and a point providing unit 1013. Each of the function modules may be implemented by executing a stored program by a CPU.

The data acquisition unit 1011 periodically obtains data transmitted from the sensor device 200. More specifically, as described above, the exercise category, exercise intensity, accumulated value of calories burned, duration of exercise, and the like are obtained for each user ID. Note that in descriptions hereinafter, the data including them is referred to as exercise data. The exercise data describes exercise details per data transmission period (for example, a unit time such as one minute). In addition, the exercise intensity is a value representing metabolic equivalents (METS).

The exercise evaluation unit 1012 evaluates the content of an exercise performed by a user, based on the data obtained from the sensor device 200. The exercise evaluation unit 1012 evaluates whether the exercise performed by the user is appropriate for the user, and generates an evaluation result. A concrete evaluation method will be described later.

The evaluation result is transmitted to the point providing unit 1013 which will be described later.

The point providing unit 1013 generates incentive data indicating points that are to be provided the user, based on the evaluation result generated by the exercise evaluation unit 1012. In this embodiment, the point providing unit 1013 provides points at a predetermined rate (for example, one yen worth of points for every 10 kilo calorie burn) when the user performs a predetermined exercise.

In addition, in this embodiment, the point providing unit 1013 lowers the point provision rate when an evaluation result generated by the exercise evaluation unit 1012 is negative, that is, when it is determined that an exercise appropriate for the user is not being performed. For example, the point providing unit 1013 lowers the point provision rate by temporarily changing a provision reference to “one yen worth of points for every 20 kilo calorie burn.”

The storage unit 102 includes a main memory and an auxiliary memory. The main memory is a memory where a program executed by the control unit 101 and data used by the control program are deployed. The auxiliary memory is a device where a program executed in the control unit 101 and data used by the control program are stored.

The storage unit 102 stores personal data 102A, evaluation data 102B, and provision reference data 102C.

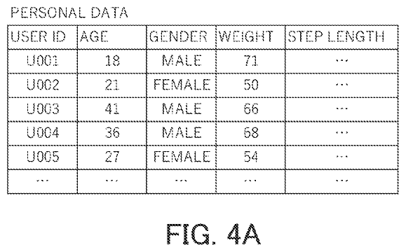

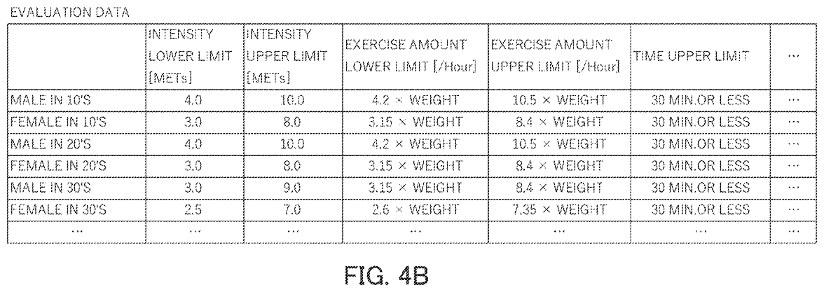

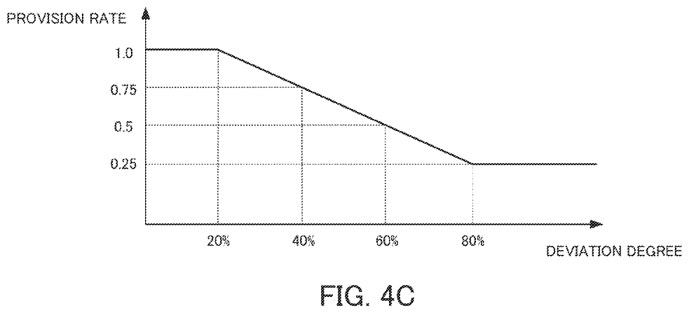

The personal data 102A is data in which a user's profile is recorded. FIG. 4A is an example of the personal data 102A. As illustrated, in this example, the age, gender, weight, and a step length of each user are stored in association with a user ID.

The evaluation data 102B is data that defines the range of exercise appropriate for each user. FIG. 4B is an example of the evaluation data 102B. For example, an appropriate exercise intensity (load applied to body), appropriate exercise amount (calorie burned per unit time) are different according to the gender, age, weight, and the like of each user. In this embodiment, the exercise evaluation unit 1012 determines whether an exercise being performed is appropriate, based on the personal data 102A and the evaluation data 102B.

In the evaluation data 102B of the illustrated example, information on the upper limit and lower limit of an exercise intensity, the upper limit and lower limit of an exercise amount (calorie burned per unit time), and the upper limit of exercise time is included.

The provision reference data 102C is data that specifies the amount of incentives to be provided to a user. The provision reference data 102C may be, for example, a table or numerical expression in which a relationship between calorie burned and points to be provided is defined. The provision reference data 102C specifies, when a user's exercise deviates from a proper range, how much the point provision rate is to be lowered. FIG. 4C is a graph that shows the provision rate of points. In this example, the point provision rate to an exercise amount (for example, calories burned) has been determined and as a deviation from a reference increases, the provision rate is lowered.

A communication unit 103 is a communication interface similar to the communication unit 203. In addition, the communication unit 103 is configured to be able to communicate with an external device that manages points via a wide-area network such as the Internet, for example.

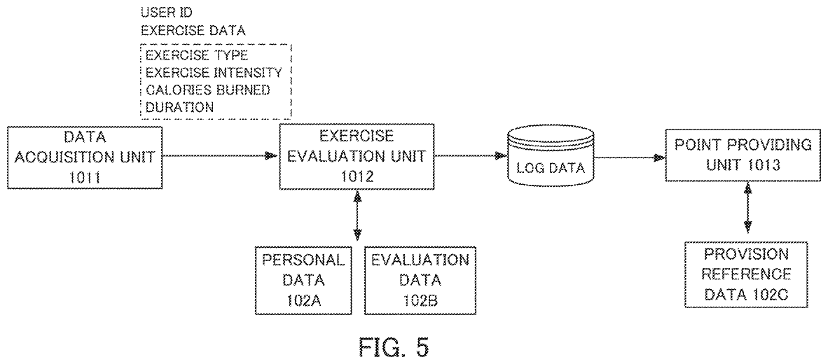

Next, a flow of processing performed by the control unit 101 will be described. FIG. 5 is a diagram that illustrates a data flow between the modules included in the control unit 101.

The data acquisition unit 1011 generates exercise data based on data obtained from the sensor device 200; and transmits to the exercise evaluation unit 1012 in association with a user ID.

The exercise evaluation unit 1012 evaluates an exercise performed by a user, based on the obtained data; and records the content of the exercise and a result of the evaluation in association with each other, in log data.

More specifically, first, it obtains the user ID included in the obtained exercise data; and extracts a corresponding record from the personal data 102A. Then, a user's profile (for example, age: 31, gender: male; weight: 66 kg) is specified. Next, based on the record, it extracts a corresponding record from the evaluation data 102B. The extracted record is used as a reference in exercise evaluation.

Thus, for example, such references are obtained as “the lower limit of exercise intensity=3.0 METs,” “the upper limit of exercise intensity=9.0 METs,” “the lower limit of calories burned per hour=3.15×66=207.9 kcal,” and “the upper limit of calories burned per hour=8.4×66=554.5 kcal.”

The exercise evaluation unit 1012 evaluates the obtained exercise data by using these references. For example, when the current intensity of an exercise is 5.0 METs and calories burned per hour are 300 kcal (which are both within their reference ranges), it determines that the exercise is appropriate. In addition, when the content of the exercise is out of the range of a reference, it calculates a value indicating how much a deviation from the reference has occurred (deviation degree). The calculated deviation degree is recorded in the log data and referred to by the point providing unit 1013.

The point providing unit 1013 refers to the provision reference data 102C and determines the provision rate of points which are to be provided to the user; and calculates its amount. For example, in a case of the example of FIG. 4C, when the deviation degree is 20% or less, the provision rate is 1.0 times its original rate and when the deviation degree is 80% or more, the provision rate is 0.25 times its original rate.

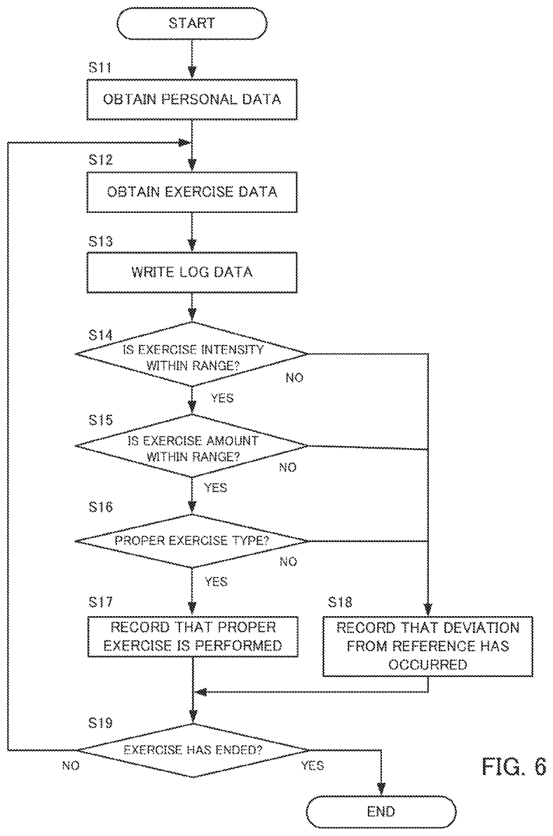

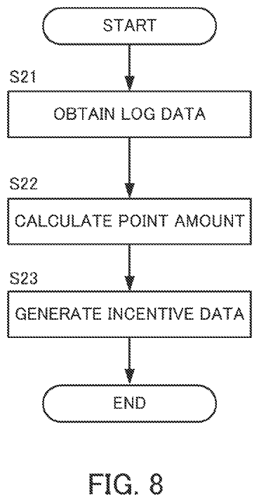

Next, processing performed by the control unit 101 will be described with reference to flowcharts. FIG. 6 is a flowchart of processing in which the control unit 101 evaluates an exercise performed by a user and generates log data including a result of the evaluation. In addition, FIG. 8 is a flowchart of processing for providing incentives to a user based on the log data.

First, at step S11, the exercise evaluation unit 1012 obtains a user ID via the data acquisition unit 1011 and obtains personal data corresponding to the user.

Next, at step S12, the exercise evaluation unit 1012 obtains exercise data via the data acquisition unit 1011.

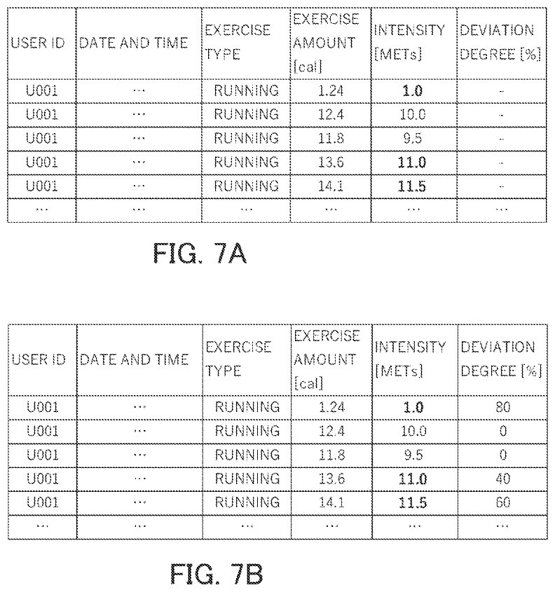

The exercise data is written in the storage unit 102 as log data at step S13. FIG. 7A is an example of log data to be written. The log data is data in which exercise performance per unit time (for example, one minute) is recorded.

The steps S14 to S16 are steps in which the exercise evaluation unit 1012 determines whether an exercise that is indicated by the exercise data obtained at step S12 is appropriate by referring to the personal data and evaluation data.

At step S14, it is determined whether the exercise intensity indicated by the exercise data is within a predetermined range. For example, if an exercise intensity (METs) indicated by the exercise data is out of a range indicated by the evaluation data, a negative determination is made.

At step S15, it is determined whether an exercise amount indicated by the exercise data is within a predetermined range. For example, if an exercise amount (calories burned per unit time) indicated by the exercise data is out of a range indicated by the evaluation data, a negative determination is made.

At step S16, it is determined whether an exercise category indicated by the exercise data is appropriate. In this step, appropriateness can be determined based on, for example, references described below:

(1) Whether the category of exercise being performed by a user is appropriate for the user

If an exercise which is inappropriate in view of the age, gender, weight, and the like of the user is performed (for example, if a load of weight lifting is too much), a negative determination is made.

(2) Whether the duration of one exercise is appropriate

For example, if the same exercise is performed over 30 minutes in a case where an upper limit time of one exercise is 30 minutes, a negative determination is made.

(3) Whether the category of an exercise performed for a past predetermined period is appropriate

For example, if a predetermined number of exercises are not performed within a predetermined period in a case where a plurality of categories of exercises have to be combined, a negative determination is made.

If an affirmative determination is made at all the steps S14 to S16, processing transitions to step S17 and the exercise evaluation unit 1012 adds an evaluation result indicating an appropriate exercise, to the log data written at step S13. FIG. 7B is an example of log data after evaluation results have been added thereto. If an appropriate exercise has been performed, for example, “0%” is recorded as a deviation degree, as illustrated.

If a negative determination is made at any one of steps S14 to S16, processing transitions to step S18 and the exercise evaluation unit 1012 adds an evaluation result indicating an inappropriate exercise, to the log data written at step S13. Here, a degree of deviation (deviation degree) from a reference may be calculated and written to the log data. The deviation degree may be, for example, one that is obtained by representing the degree of deviation with a numeric value from 0 to 100. When an evaluation result indicates that there is a deviation from a reference, a value equal to or higher than 1% is recorded as the deviation degree, as illustrated in FIG. 7B. A method of calculating the deviation degree is not limited to a specific one.

At step S19, whether the exercise has ended is determined. If the exercise has not ended, the processing returns to step S12. If the exercise has ended (for example, if a notification that the exercise ends is transmitted from the sensor device 200), the processing ends. Note that if data has not been transmitted from the sensor device 200 for a predetermined time or more, the processing may be ended.

Through the above processing, whether the content of an exercise being performed by the user is appropriate is periodically evaluated and recorded.

FIG. 8 is a flowchart of processing for calculating the amount of points which are to be provided to a user by the point providing unit 1013, based on recorded log data. This processing may be executed while the user is performing an exercise, or may be executed after the exercise has ended.

First, at step S21, target log data is obtained. In this step, for example, records based on which incentives have not been provided to a target user are extracted from the log data.

Next, at step S22, the amount of points for each obtained record is calculated. The amount of points is calculated based on a recorded deviation degree and the provision reference data 102C.

Here, one yen worth of points are provided for every 10 kilo calories as a standard. In an example shown in FIG. 4C, when the provision rate is 1.0, one yen worth of points per 10 kilo calories are provided. In addition, when the provision rate is 0.5, 0.5-yen worth points are provided.

For example, in a case where the exercise amount per one minute is 12.4 kilo calories and the deviation degree is 20% or less (that is, the provision rate is 1.0), the point providing unit 1013 determines to provide (12.4/10)×1.0=1.24 yen worth of points to the user.

In addition, in a case where the exercise amount per one minute is 14.1 kilo calories and the deviation degree is 60% (that is, the provision rate is 0.5), the point providing unit 1013 determines to provide (14.1/10)×0.5=0.705 yen worth of points to the user.

Next, at step S23, the amounts of points determined for individual records are totalized to generate incentive data. The generated incentive data is transmitted to an external device that manages points.

As described above, the evaluation device 100 according to the first embodiment determines whether an exercise being performed by a user is appropriate based on data obtained from the sensor device 200 and if it is not appropriate, reduces the amount of incentives to be provided to the user. This configuration can prevent a user from repeating an inappropriate exercise in order to obtain incentives.

(Modification of First Embodiment)

In the first embodiment, exercise evaluation is performed by using an exercise intensity, exercise amount, and duration; however, for a training menu including a plurality of exercises, exercise evaluation may be performed further based on the achievement degree of the menu. For example, when an exercise which is not included in the menu is performed, the deviation degree may be set higher. In addition, when a next exercise is not started even though a goal has been attained, when exercise ends with a specified menu not executed, or when exercise does not end after a target time has passed, the deviation degree may be set higher.

### Second Embodiment

In a second embodiment, when it is determined that exercise is not appropriately performed, a user is notified to that effect and is encouraged to perform an improvement.

In the second embodiment, the communication unit 103 includes a function of performing wireless communication with a portable terminal (user terminal) carried by a user. The user terminal may also be used as a wearable computer as the sensor device 200.

In addition, in the second embodiment, when determining that a deviation from an evaluation reference has occurred in an exercise evaluation process, the exercise evaluation unit 1012 notifies the user terminal to that effect. More specifically, it generates a notification including both an evaluation reference (for example, an exercise intensity or exercise amount) from which the deviation has occurred and a concrete value, and transmits it to the user terminal. In this case, information on the incentive provision rate may be included.

Thus, for example, specific advice such as “to lower the exercise intensity,” “to reduce the exercise amount,” or “to execute a different exercise menu” can be given to the user.

According to the second embodiment, notification that an inappropriate exercise is being performed can be provided to the user in real time. This can prevent overworking and encourage well-balanced exercise.

(Modification)

The above embodiments are merely one example, and the present disclosure can be appropriately modified and implemented without departing from the spirit thereof.

For example, the processing and units described in the present disclosure can be implemented in combination as long as a technical contradiction does not occur.

In addition, in the description of the embodiments, points that can be used in online services are provided as examples of incentives; however, incentives may be others. For example, they may be virtual currencies, discount tickets, or the like; preferential treatment (for example, a preferential treatment for an insurance premium rate) in services related to health conditions (for example, life insurance); or the like.

In addition, in the description of the embodiments, when an exercise content deviates from a proper range for a user, incentives are reduced; however, on the contrary, when an exercise content is a proper one for a user, incentives may be increased. For example, when a deviation from evaluation references has not occurred for a predetermined number of days or more, a bonus may be provided.

In addition, the processing described as being performed by one device may be shared and executed by a plurality of devices. Alternatively, the processing described as being performed by different devices may be executed by one device. In a computer system, what hardware configuration (server configuration) realizes each function can be flexibly changed.

The present disclosure can also be realized by supplying a computer program including the functions described in the above embodiments to a computer and causing one or more processors included in the computer to read and execute the program. Such a computer program may be provided to the computer by a non-transitory computer-readable storage medium connectable to a system bus of the computer, or may be provided to the computer via a network. Examples of non-transitory computer readable storage media include: any type of disk such as a magnetic disk (floppy (registered trademark) disk, hard disk drive (HDD), etc.), an optical disk (CD-ROM, DVD disk, Blu-ray disk, etc.); and any type of medium suitable for storing electronic instructions, such as read-only memory (ROM), random access memory (RAM), EPROM, EEPROM, magnetic cards, flash memory, and optical cards.

What is claimed is:

1. An evaluation device, comprising:
a controller configured to:
obtain exercise data, the exercise data being data related to exercise performed by a user;
generate incentive data based on the exercise data, the incentive data indicating incentives to be provided to the user;
determine whether an exercise appropriate for the user is being performed, the determining being performed based on the exercise data; and
when determining that an exercise appropriate for the user is not performed, adjust an amount of the incentives to be provided to the user.

2. The evaluation device according to claim 1, further comprising:
a storage configured to store evaluation data for evaluating the exercise performed by the user; wherein
the controller determines, based on the evaluation data, whether at least any one of an exercise intensity, an exercise amount, and an exercise type is appropriate for the user.

3. The evaluation device according to claim 2, wherein
the storage further stores user information, the user information being information about the user; and
the controller determines an exercise intensity, exercise amount, or exercise type that is appropriate for the user, further based on the user information.

4. The evaluation device according to claim 2, wherein
when determining that the exercise intensity or the exercise amount deviates from a proper range for the user, the controller reduces a provision rate of the incentives to be provided to the user.

5. The evaluation device according to claim 2, wherein
when determining that the exercise type is not appropriate for the user, the controller reduces a provision rate of the incentives to be provided to the user.

6. The evaluation device according to claim 5, wherein
when determining that a type of exercise performed for a past predetermined period is not appropriate for the user, the controller reduces a provision rate of the incentives to be provided to the user.

7. The evaluation device according to claim 1, wherein
when determining that an exercise appropriate for the user is not being performed, the controller notifies the user that the amount of the incentives is to be adjusted.

8. The evaluation device according to claim 7, wherein
when determining that an exercise appropriate for the user is not being performed, the controller suggests an exercise menu to the user, the exercise menu allowing the adjustment to be avoided.

9. An evaluation system, comprising:
one or more sensors configured to obtain exercise data, the exercise data being data related to an exercise performed by a user; and
a controller configured to:
generate incentive data based on the exercise data, the incentive data indicating incentives to be provided to the user;
determine, based on the exercise data, whether an exercise appropriate for the user is being performed; and
when determining that an exercise appropriate for the user is not performed, adjust an amount of the incentives to be provided to the user.

10. The evaluation system according to claim 9, further comprising:
a storage configured to store evaluation data, the evaluation data for evaluating an exercise performed by the user; wherein
the controller determines, based on the evaluation data, whether at least any one of an exercise intensity, an exercise amount, and an exercise type is appropriate for the user.

11. The evaluation system according to claim 10, wherein
the one or more sensors include a sensor built in a wearable computer associated with the user.

12. The evaluation system according to claim 11, wherein
the one or more sensors include a heart rate sensor; and
the exercise data includes an exercise intensity estimated based on a heart rate, the heart rate being obtained by the heart rate sensor.

13. The evaluation system according to claim 10, wherein
the storage further stores user information, the user information being information about the user; and
the controller determines an exercise intensity, an exercise amount, or an exercise type that is appropriate for the user, further based on the user information.

14. The evaluation system according to claim 10, wherein
when determining that the exercise intensity or the exercise amount deviates from a proper range for the user, the controller reduces a provision rate of the incentives to be provided to the user.

15. The evaluation system according to claim 10, wherein
when determining that the exercise type is not appropriate for the user, the controller reduces a provision rate of the incentives to be provided to the user.

16. The evaluation system according to claim 15, wherein
when determining that a type of exercise performed for a past predetermined period is not appropriate for the user, the controller reduces the provision rate of the incentives to be provided to the user.

17. The evaluation system according to claim 9, wherein
when determining that an exercise appropriate for the user is not being performed, the controller notifies the user that the amount of the incentives is to be adjusted.

18. The evaluation system according to claim 17, wherein
when determining that an exercise appropriate for the user is not being performed, the controller suggests an exercise menu to the user, the exercise menu allowing the adjustment to be avoided.

19. A non-transitory computer readable storing medium recording a computer program for causing a computer to perform an information processing method comprising:
a step of obtaining exercise data, the exercise data being data related to an exercise performed by a user;
a step of generating incentive data based on the exercise data, the incentive data indicating incentives to be provided to the user;
a step of determining, based on the exercise data, whether an exercise appropriate for the user is being performed; and
a step of, when determining an exercise appropriate for the user is not being performed, adjusting an amount of the incentives to be provided to the user.

20. The non-transitory storage medium according to claim 19, wherein:
the program further causes the computer to perform:
a step of, when determining that an exercise appropriate for the user is not being performed, reducing a provision rate of the incentives to be provided to the user.
